(19) United States
(12) Patent Application Publication
(10) Pub. No.: US 2025/0116642 A1
(43) Pub. Date: Apr. 10, 2025

(54) MEASURING SYSTEM OF GASEOUS SUBSTANCES

(71) Applicant: Hongik University Industry-Academia Cooperation Foundation, Seoul (KR)

(72) Inventor: Jung Hwan Seo, Seoul (KR)

(21) Appl. No.: 18/721,601

(22) PCT Filed: Dec. 28, 2022
(86) PCT No.: PCT/KR2022/021459

§ 371 Date: Jun. 18, 2024

(30) Foreign Application Priority Data
Dec. 30, 2021 (KR) 10-2021-0192770
Dec. 19, 2022 (KR) 10-2022-0178317

(51) Int. Cl. G01N 33/00; G01N 27/66

(52) U.S. Cl. CPC G01N 33/0021; G01N 27/66

(57) ABSTRACT

A measuring system for metering components of a gas mixture including gaseous phase materials has a sampling unit configured to selectively communicate with a pipe through which the gas mixture passes to sample the gas mixture from the pipe, a detection unit configured to separate and detect the gaseous phase materials included in the gas mixture sampled by the sampling unit into the components, a valve assembly including a plurality of valves to selectively bring the sampling unit into communication with the pipe or the detection unit. The plurality of valves cooperates to selectively perform a sampling connection operation of bringing the pipe and the sampling unit into communication with each other and a sampling interruption operation of interrupting the communication between the pipe and the sampling unit.

### CROSS-REFERENCE TO RELATED APPLICATIONS

This application is a National Stage application under 35 U.S.C. § 371 of International Application No. PCT/KR2022/021459, having an International Filing Date of Dec. 28, 2022, which claims priority to South Korean Application Serial No. 10-2021-0192770, filed on Dec. 30, 2021, and South Korean Application Serial No. 10-2022-0178317, filed on Dec. 19, 2022. The disclosure of the prior applications is considered part of the disclosure of this application, and is incorporated in its entirety into this application.

### TECHNICAL FIELD

The present disclosure relates to a measuring system, and more particularly, to a measuring system for metering the components of a gas mixture including gaseous phase materials flowing in a pipe.

### BACKGROUND

In a semiconductor fabrication process, surface treatment of semiconductor components such as wafers, for example, cleaning, etching or the like, occupies a very important position in ensuring the yield and accuracy of products. In general, the cleaning and etching processes of the semiconductor components are performed using plasma and UV ozone.

To identify the state of the wafer to determine whether the process was properly completed, after the process is completed, the substances on the wafer surface are analyzed by a wafer surface substance analyzer of high price (a few hundreds of millions to a few billions) using Time of Flight (ToF)-Secondary Ion Mass Spectroscopy (SIMS).

The TOF-SIMS method is used to identify the chemical composition and surface structure by analyzing cations or anions emitted while primary ions strike a surface, and has a wide detection area and high precision, but an instrument for using the corresponding method is expensive and cannot be reduced in size, thereby failing to detect in situ. Additionally, to use the TOF-SIMS method, after the process of the semiconductor component is completed, it is necessary to test the semiconductor component, and thus real-time monitoring is impossible.

Accordingly, in practice, it is impossible to monitor the current status of the process in real time during the process and control the process in a personalized optimal manner.

A Residual Gas Analyzer (RGA) is used to monitor the status of the semiconductor process in real time during the process. However, RGA is also very expensive, fails to analyze gas mixtures, and only works in a high vacuum chamber. The RGA has a limited use in deposition applications.

Due to the technical and economic reasons, in the main process of semiconductor treatment such as etching or cleaning, during the process, there is no apparatus for real-time (in-situ) measurement to identify the presence or absence of the remaining materials on the surface of the semiconductor component, and the controlled state and leakage of gaseous phase materials used in the corresponding process.

In a series of processes requiring real-time analysis of gaseous phase materials like a semiconductor process, there is a need for development of a measuring system for easily and appropriately metering the components of a gas mixture produced as a result of the process.

### SUMMARY

The present disclosure provides a measuring system for metering the components of a gas mixture including gaseous phase materials produced through a series of processes by directly sampling the gas mixture from a pipe.

According to an aspect of the present disclosure, there is provided a measuring system for metering components of a gas mixture including gaseous phase materials, including: a sampling unit configured to selectively communicate with a pipe through which the gas mixture passes to sample the gas mixture from the pipe, a detection unit configured to separate and detect the gaseous phase materials included in the gas mixture sampled by the sampling unit into the components, a valve assembly including a plurality of valves to selectively bring the sampling unit into communication with the pipe or the detection unit, wherein the plurality of valves cooperates to selectively perform a sampling connection operation of bringing the pipe and the sampling unit into communication with each other and a sampling interruption operation of interrupting the communication between the pipe and the sampling unit.

According to an embodiment, simultaneously with the sampling interruption operation or after the sampling interruption operation, the measuring system performs a gas delivery operation of forming a flow channel to allow a carrier gas to flow with the gas mixture by cooperation of the plurality of valves.

According to an embodiment, the flow channel is formed by the gas delivery operation to allow the carrier gas to flow to the detection unit through the sampling unit.

According to an embodiment, in the sampling connection operation, the channel of fluid flow from a carrier gas tank directly to the detection unit and the channel of fluid flow from the pipe back to the pipe through the sampling unit are formed, and in the gas delivery operation, the channel of fluid flow from the carrier gas tank to the detection unit through the sampling unit is formed.

According to an embodiment, the sampling interruption operation includes a sampling route closing operation of trapping the gas mixture in the sampling unit, and a depressurization operation of reducing a pressure of the gas mixture trapped in the sampling unit.

According to an embodiment, the detection unit includes a depressurization chamber to reduce the pressure of the gas mixture, the gas mixture is allowed to enter the depressurization chamber to reduce the pressure through the depressurization operation, and after the pressure of the gas mixture is reduced, the gas delivery operation is performed.

According to an embodiment, the plurality of valves includes a plurality of multi-port valves, each having a plurality of ports, and the plurality of multi-port valves selectively operates to selectively perform the sampling connection operation, the sampling interruption operation and the gas delivery operation.

According to an embodiment, the plurality of multi-port valves is a manual valve that switches the fluid channel in the valve by a lever that is manually manipulated, each lever of the plurality of multi-port valves is connected to a link structure together, and the levers of the plurality of multi-port valves are simultaneously operated by operation of the link structure to simultaneously switch the fluid channels.

According to an embodiment, the plurality of multi-port valves includes first to third 3-way valves, each having three ports, wherein a first port of the first 3-way valve is connected to the pipe, a second port is connected to an end of a first connection pipe, and a third port is connected to the pipe, wherein a first port of the second 3-way valve is connected to an entrance of the sampling unit, a second port is connected to an opposite end of the first connection pipe, and a third port is connected to an end of a second connection pipe, wherein a first port of the third 3-way valve is connected to an exit of the sampling unit, a second port is connected to the pipe, and a third port is connected to an end of a third connection pipe, and wherein the sampling unit and the pipe are brought into communication with each other or the communication is interrupted by operation of the first 3-way valve.

According to an embodiment, the plurality of multi-port valves includes a fourth 3-way valve and a fifth 3-way valve, each having three ports, wherein a first port of the fourth 3-way valve is connected to a carrier gas inlet pipe that communicates with a carrier gas tank, a second port is connected to an opposite end of the second connection pipe, and a third port is connected to an end of a fourth connection pipe, wherein a first port of the fifth 3-way valve is connected to a detection unit inlet pipe that communicates with the detection unit, a second port is connected to an opposite end of the fourth connection pipe, and a third port is connected to an opposite end of the third connection pipe, and wherein the first to fifth 3-way valves selectively operate to switch the fluid channel to selectively perform the sampling connection operation, the sampling interruption operation and the gas delivery operation.

According to an embodiment, after the pressure of the gas mixture is reduced through the sampling interruption operation, the gas delivery operation is performed, wherein the sampling interruption operation includes the sampling route closing operation of trapping the gas mixture in the sampling unit and the depressurization operation of allowing the gas mixture trapped in the sampling unit to flow to the depressurization chamber to reduce the pressure of the gas mixture, wherein in the sampling route closing operation, the first 3-way valve and the third 3-way valve simultaneously operate to switch the fluid channel, and wherein in the depressurization operation, the fifth 3-way valve operates to switch the fluid channel.

According to an embodiment, in the gas delivery operation, the second 3-way valve and the fourth 3-way valve simultaneously operate to switch the fluid channel.

According to an embodiment, the gas delivery operation is performed simultaneously with the sampling interruption operation, and the first to fifth 3-way valves simultaneously operate to switch the fluid channel to simultaneously perform the sampling interruption operation and the gas delivery operation.

According to an embodiment, the plurality of multi-port valves includes a first 4-way valve and a second 4-way valve, each having four ports, wherein a first port of the first 4-way valve is connected to a carrier gas inlet pipe that communicates with a carrier gas tank, a second port is connected to an entrance of the sampling unit, a third port is connected to the pipe, and a fourth port is connected to an end of a fifth connection pipe, wherein a first port of the second 4-way valve is connected to a detection unit inlet pipe that communicates with the detection unit, a second port is connected to an exit of the sampling unit, a third port is connected to the pipe, and a fourth port is connected to an opposite end of the fifth connection pipe, wherein the first 4-way valve and the second 4-way valve simultaneously operate to switch the fluid channel, wherein in the sampling connection operation, the channel of fluid flow from the carrier gas tank directly to the detection unit and the channel of fluid flow from the pipe back to the pipe through the sampling unit are formed, and wherein in the gas delivery operation, the channel of fluid flow from the carrier gas tank to the detection unit through the sampling unit is formed.

According to an embodiment, the first 4-way valve and the second 4-way valve are an automatic valve that automatically operates by control of a controller.

### BRIEF DESCRIPTION OF THE DRAWINGS

FIG. 1 is a schematic system diagram of a measuring system according to an embodiment.

FIG. 2 is a schematic diagram of a valve assembly according to Embodiment 1.

FIG. 3 is a schematic diagram showing the arrangement of each valve of the valve assembly of FIG. 2 in three dimensions.

FIGS. 4 and 5 show a state of the valve assembly of FIGS. 2 and 3 in a sampling connection operation.

FIGS. 6 and 7 show a state of the valve assembly of FIGS. 2 and 3 in a sampling route closing operation while in a sampling interruption operation.

FIGS. 8 and 9 show a state of the valve assembly of FIGS. 2 and 3 in a depressurization operation while in a sampling interruption operation.

FIGS. 10 and 11 show a state of the valve assembly of FIGS. 2 and 3 in a gas delivery operation.

FIG. 12 is a schematic system diagram of a measuring system according to a variation of Embodiment 1.

FIG. 13 is a schematic diagram of a valve assembly according to a variation of Embodiment 1.

FIGS. 14 and 15 are schematic diagrams of a valve assembly according to Embodiment 2; FIG. 14 shows a state in a sampling connection operation, and FIG. 15 shows a state in a sampling connection operation.

FIG. 16 is a schematic conceptual view of a detection device according to an embodiment of the present disclosure.

FIG. 17 is an enlarged view of a concentration module formed in the detection device of FIG. 16.

FIG. 18 is a bottom view of a substrate coupled to the detection device of FIG. 16.

FIG. 19 schematically shows the inside of a separation module formed in the detection device of FIG. 16.

FIG. 20 schematically shows a separation and detection process of gaseous phase materials using the detection device of FIG. 16.

FIG. 21 is a graph showing a result of detection through a measuring system according to an embodiment.

### DETAILED DESCRIPTION

Hereinafter, exemplary embodiments of the present disclosure will be described with reference to the accompanying drawings. The present disclosure is described with reference to the embodiments shown in the drawings, but the description is provided as an embodiment and the technical spirit of the present disclosure and its essential components and operation are not limited thereto.

“Upstream” as used herein refers to a side from which a fluid flows, and “downstream” refers to an opposite side to the upstream.

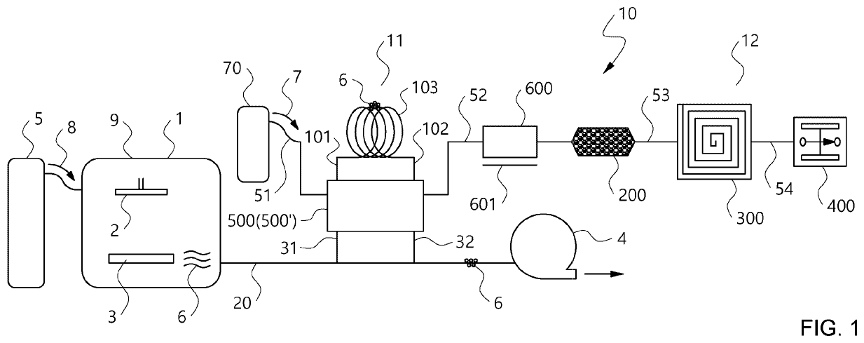

FIG. 1 is a schematic system diagram of a measuring system 10 according to an embodiment of the present disclosure.

The measuring system 10 according to this embodiment is a measuring system for detecting the status of a process for a component in a treatment apparatus for treating the component in a chamber to generate a gas mixture 6 including gaseous phase materials 60.

### Treatment Apparatus 1

The treatment apparatus 1 according to this embodiment is a semiconductor treatment apparatus that performs a plasma cleaning process to clean a wafer 3 which is the semiconductor component through a plasma generator (electrode) 2 in the chamber 9 in a vacuum state.

When the plasma cleaning process starts, a cleaning gas 8 (for example, O₂) is fed into the chamber 9 in the vacuum state from an external cleaning gas tank 5.

The cleaning gas 8 ionized by the plasma generated by the plasma generator 2 reacts with compounds of the surface of the wafer 3 to produce the gaseous phase materials 60 which are organic compounds of different substances.

A gas mixture 6 including gaseous phase materials 60 is discharged through a pipe 20 that communicates with a chamber 9. A pump 4 is connected to the downstream side of the pipe 20 to form the vacuum in the chamber 9 and provide the pressure for discharging the gas mixture 6.

The configuration of the semiconductor treatment apparatus for performing the plasma cleaning process is known, and its detailed description is omitted.

### Measuring System 10

The measuring system 10 according to this embodiment is configured to detect the gaseous phase materials 60 included in the gas mixture 6 substantially in real time by analyzing the gas mixture 6 discharged from the treatment apparatus 1.

Referring to FIG. 1, the measuring system 10 includes a sampling unit 11 that selectively communicates with the pipe 20 of the treatment apparatus 1 to sample a predetermined volume of the gas mixture 6 from the pipe 20 for a predetermined time, and a detection unit 12 to separate and detect the gaseous phase materials 60 included in the gas mixture 6 sampled by the sampling unit 11. Additionally, the measuring system 10 includes a valve assembly 500 to selectively bring the sampling unit 11 into communication with the pipe 20 or the detection unit 12.

The sampling unit 11 according to this embodiment includes an inlet 101 through which the gas mixture 6 enters, an outlet 102 through which the gas mixture 6 exits, and a sampler module 103 between the inlet 101 and the outlet 102.

The inlet 101 and the outlet 102 have a long conduit shape, and the sampler module 103 has a spirally coiled shape of a conduit shape.

Here, the sampler module 103 may have any other shape, and for convenience of description, the inlet 101 and the outlet 102 are different from each other, but the sampling unit 11 according to this embodiment is formed by spirally winding the intermediate portion of a single conduit.

Since the sampler module 103 is formed in a spiral shape, it is possible to increase the volume of the gas mixture 6 sampled by the sampling unit 11.

The sampling unit 11 according to this embodiment has the conduit shape with two open ends, and the gas mixture 6 continuously flows in the conduit while the sampling unit 11 is in communication with the pipe 20. That is, the gas mixture 6 of the same volume is always filled in the sampling unit 11.

Accordingly, when the sampling unit 11 and the pipe 20 are in non-communication with each other, the gas mixture 6 of the same volume is always sampled in the sampling unit 11 at any time.

As shown in FIG. 1, an upstream connection pipe 31 and a downstream connection pipe 32 are branched from the pipe 20. According to this embodiment, the diameter of the upstream connection pipe 31 and the downstream connection pipe 32 is substantially the same as the diameter of the sampling unit 11.

The sampling unit 11 is selectively brought into communication with the upstream connection pipe 31 and the downstream connection pipe 32 and selectively brought into communication with the pipe 20 by the valve assembly 500.

According to this embodiment, the upstream connection pipe 31 and the downstream connection pipe 32 are connected to the pipe 20 between the chamber 9 and a pump 4. That is, the sampling unit 11 is in communication with the pipe 20 at the upstream side of the pump 4.

Accordingly, it may be possible to prevent the sampled gas mixture 6 from being contaminated with oils from the pump 4. Furthermore, the measuring system 10 may be easily applied to the existing treatment apparatus 1 by modifying the pipe 20 that is relatively easy to change the structure between the chamber 9 and the pump 4, so that the upstream connection pipe 31 and the downstream connection pipe 32 are connected to the pipe 20.

The valve assembly 500 according to this embodiment includes a plurality of valves and connection pipes connecting the valves. FIG. 1 simply shows the valve assembly 500 in a rectangular block shape for convenience of illustration, and an embodiment of detailed configuration will be described below.

According to this embodiment, the plurality of valves of the valve assembly 500 cooperates with each other to selectively perform a sampling connection operation of bringing the pipe 20 and the sampling unit 11 into communication with each other and a sampling interruption operation of interrupting the communication between the pipe 20 and the sampling unit 11.

The valve assembly 500 is connected to the upstream connection pipe 31 and the downstream connection pipe 32, and when necessary, forms a flow channel to allow the gas mixture 6 flowing in the pipe 20 to return to the pipe 20 through the valve assembly 500.

Also, the valve assembly 500 is connected to an entrance 101 of the sampling unit 11, and connected to an exit 102 of the sampling unit 11.

Also, the valve assembly 500 is connected to a carrier gas inlet pipe 51 connected to a carrier gas tank 70, and connected to a detection unit inlet pipe 52 leading to the detection unit 12.

The detection unit 12 according to this embodiment includes a depressurization chamber 600 to accommodate the gas mixture 6 sampled by the sampling unit 11 and reduce the pressure of the gas mixture 6, a concentration module 200 to filter, concentrate and store the gaseous phase materials 60 included in the gas mixture 6 subjected to depressurization in the depressurization chamber 600, a separation module 300 to separate the gaseous phase materials 60 concentrated by the concentration module 20 into components, and a sensor module 400 to detect the gaseous phase materials 60 from the separation module 300. The depressurization chamber 600, the concentration module 200, the separation module 300 and the sensor module 400 are in communication with each other by conduits 52, 53, 54. Each module of the detection unit 12 will be described in more detail below.

### Operation of the Measuring System 10

Hereinafter, the operation of the measuring system 10 for measuring the gaseous phase materials 60 will be described with reference to FIG. 1.

As shown in FIG. 1, when a plasma cleaning process starts by the treatment apparatus 1, the valve assembly 500 performs a sampling connection operation to sample the gas mixture 6 by the sampling unit 11.

More specifically, in the sampling connection operation, the valve assembly 50 operates to form a channel of fluid flow from the carrier gas tank 70 directly to the detection unit 12 and a channel of fluid flow from the pipe 20 back to the pipe 20 through the sampling unit 11.

According to this embodiment, in the sampling connection operation (other than a gas delivery operation as described below), the channel of fluid flow from the carrier gas tank 70 directly to the detection unit 12 is formed (i.e., the carrier gas tank 70 and the detection unit 12 are in communication with each other) to allow carrier gas (for example, N₂ or He) 7 to flow from the carrier gas tank 70 toward the detection unit 12. Accordingly, a clean atmosphere in which the detection unit 12 is not contaminated by the outdoor air is maintained by the carrier gas 7. The carrier gas, for example, helium has very low reactivity with a porous polymer or an organic compound as described below.

When the predetermined time passes, the valve assembly 500 performs a sampling interruption operation not to perform sampling any longer.

In the sampling interruption operation, the valve assembly 500 interrupts the communication between the sampling unit 11 and the pipe 20 to trap a set volume of the gas mixture 6 in the sampling unit 11. In this embodiment, in the sampling connection operation, the channel of fluid flow from the carrier gas tank directly to the detection unit and the channel of fluid flow from the pipe back to the pipe through the sampling unit are formed.

Additionally, the valve assembly 500 performs the gas delivery operation after the sampling interruption operation or simultaneously with the sampling interruption operation.

The gas delivery operation is performed to form the flow channel to allow the carrier gas to flow in at least the analysis unit 12 together with the gas mixture 6, and in this embodiment, in the gas delivery operation, the channel of fluid flow from the carrier gas tank 70 to the detection unit 12 through the sampling unit 11 is formed.

On the other hand, in the sampling interruption operation, the valve assembly 500 forms the channel of fluid flow to allow parts of the gas mixture 6 flowing in the pipe 20 to flow back to the pipe 20 through the upstream connection pipe 31, the valve assembly 500 and the downstream connection pipe 32.

According to this embodiment, the diameter of the bypass pipe 31 and the conduit diameter of the sampling unit 11 are substantially equal to each other, and even though the sampling connection operation state is instantaneously changed to the sampling interruption operation state, there are almost no changes in volume of the gas mixture 6 escaping from the pipe 20 to the upstream connection pipe 31.

When the bypass path is suddenly blocked while the constant amount of the gas mixture 6 is bypassed and diverted from the pipe 20, shocks by the pressure may occur in the pipe 20. The pump 4 or the treatment apparatus 1 may be adversely affected by the pressure shocks.

According to this embodiment, even though the sampling connection operation state is instantaneously changed to the sampling interruption operation state, the predetermined volume of gas mixture 6 is still kept bypassed from the pipe 20 through the flow channel that passes through the upstream connection pipe 31, the valve assembly 500 and the downstream connection pipe 32, thereby suppressing an abrupt pressure change.

Furthermore, there is no need to stop the operation of the treatment apparatus 1 in the entire process of sampling the gas mixture 6, thereby increasing the yield of the semiconductor component.

In the gas delivery operation, the gas mixture 6 sampled by the sampling unit 11 may be allowed to flow to the detection unit 12 by a pump (not shown) installed at the downstream of the detection unit 12, but according to this embodiment, in the gas delivery operation, the entrance 101 of the sampling unit 11 is in communication with the carrier gas tank 70 by the valve assembly 500. The sampled gas mixture 6 flows to the detection unit 12 together with the carrier gas 7 from the carrier gas tank 70.

The gas mixture 6 flows to the concentration module 200, and the gaseous phase materials 60 included in the gas mixture 6 is filtered by the concentration module 200 to make concentrates which are stored in the concentration module 200.

Subsequently, when energy, for example, heat is applied to the concentration module 200, the gaseous phase materials 60 is discharged from the concentration module 200, and the carrier gas 7 carries the gaseous phase materials 60 to the separation module 300. The separation module 300 separates the gaseous phase materials 60 into components and discharges it.

Subsequently, the carrier gas 7 of a constant rate carries the gaseous phase materials 60 from the separation module 300 toward the sensor module 400. When the gaseous phase materials 60 reaches the sensor module 400, the sensor module 400 detects the gaseous phase materials 60.

### Valve Assembly 500, 500′

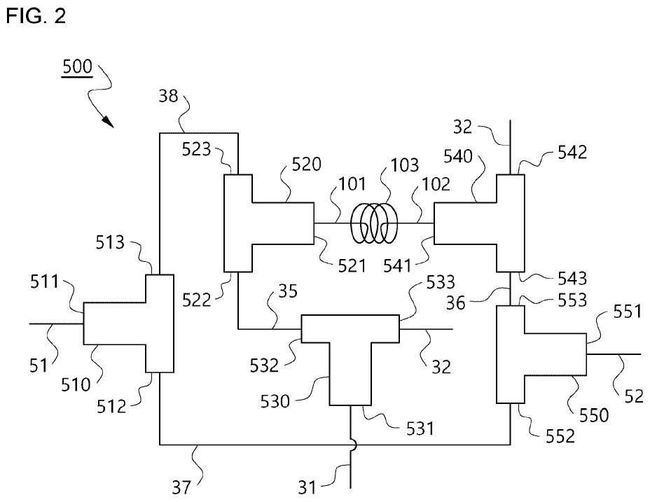

Hereinafter, embodiments of the valve assembly 500, 500′ will be described in detail with reference to FIGS. 2 to 15.

### Embodiment 1

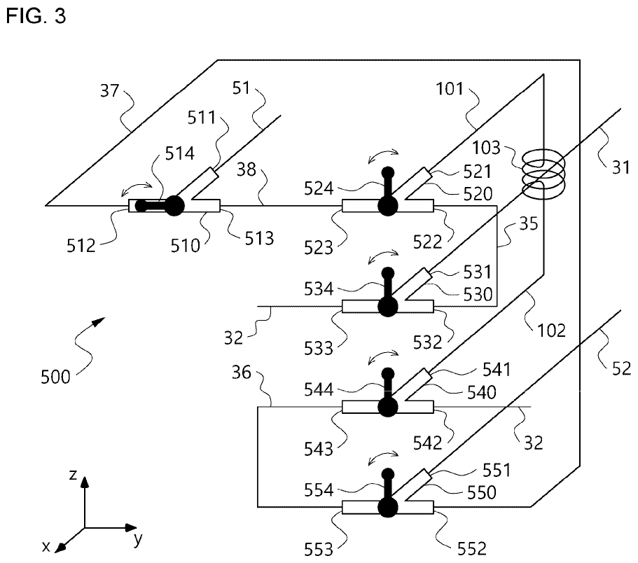

FIG. 2 is a schematic diagram of the valve assembly 500 according to an embodiment, and FIG. 3 is a schematic diagram showing the arrangement of each valve of the valve assembly 500 of FIG. 2 in three dimensions.

As shown in FIGS. 2 and 3, the valve assembly 500 according to this embodiment includes five 3-way valves, each having three ports.

A first port 531 of a first 3-way valve 530 is connected to the upstream connection pipe 31 and connected to the pipe 20, and a second port 532 is connected to an end of a first connection pipe 35, and a third port 533 is connected to the downstream connection pipe 32 and connected to the pipe 20.

A first port 521 of a second 3-way valve 520 is connected to the entrance 101 of the sampling unit 11, a second port 522 is connected to an opposite end of the first connection pipe 35 and connected to the first 3-way valve 530, and a third port 523 is connected to an end of a second connection pipe 38.

A first port 541 of a third 3-way valve 540 is connected to the exit 102 of the sampling unit 11, a second port 542 is connected to the downstream connection pipe 32 and connected to the pipe 20, and a third port 543 is connected to an end of a third connection pipe 36.

A first port 511 of a fourth 3-way valve 510 is connected to the carrier gas inlet pipe 51 that communicates with the carrier gas tank 70, a second port 512 is connected to an end of a fourth connection pipe 37, and a third port 513 is connected to an opposite end of the second connection pipe 38 and connected to the second 3-way valve 520.

A first port 551 of a fifth 3-way valve 550 is connected to the detection unit inlet pipe 52 that communicates with the detection unit 12, a second port 552 is connected to an opposite end of the fourth connection pipe 37 and connected to the fourth 3-way valve 510, and a third port 553 is connected to an opposite end of the third connection pipe 36 and connected to the third 3-way valve 540.

As shown in FIG. 3, in this embodiment, the fourth 3-way valve 510 and the second 3-way valve 520 are arranged in the horizontal direction (y direction), and the second 3-way valve 520, the first 3-way valve 530, the third 3-way valve 530 and the fifth 3-way valve 550 are arranged side by side in the vertical direction (z direction) in that order. However, the arrangement of the valves is not limited to this embodiment. As the valve assembly 500 according to this embodiment includes the plurality of valves, it is free to change the arrangement of each valve. Accordingly, when installing the measuring system 10 at the pipe 20 of the existing treatment apparatus 1, it is possible to avoid pipes or devices near the existing treatment apparatus 1, thereby forming the valve assembly 500 appropriately and effectively.

Although FIG. 3 shows that the directions of the first to third ports of each 3-way valve are the same, the orientation of the 3-way valves may be free depending on the situation of the installation space.

According to this embodiment, each 3-way valve of the valve assembly 500 is a manual valve that switches a fluid channel in the valve by the manual manipulation by a lever.

The “manual valve” refers to a manual device that switches the channel of fluid flow by the lever that is manipulated by the application of an external force to the lever, and is different from an “automatic valve” that automatically switches the channel of fluid flow by the operation of an actuator inside the valve by an electrical signal.

The first 3-way valve 530 has a handle-type lever 534 in the middle of a T-shaped body. According to the rotational direction of the lever 534, two flow channels are selectively formed; one from the first port 531 to the second port 532 and the other from the first port 531 to the third port 533.

The second 3-way valve 520 has a handle-type lever 524 in the middle of a T-shaped body. According to the rotational direction of the lever 524, two flow channels are selectively formed; one from the first port 521 to the second port 522 and the other from the first port 521 to the third port 523.

The third 3-way valve 540 has a handle-type lever 544 in the middle of a T-shaped body. According to the rotational direction of the lever 544, two flow channels are selectively formed; one from the first port 541 to the second port 542 and the other from the first port 541 to the third port 543.

The fourth 3-way valve 510 has a handle-type lever 514 in the middle of a T-shaped body. According to the rotational direction of the lever 514, two flow channels are selectively formed; one from the first port 511 to the second port 512 and the other from the first port 511 to the third port 513.

The fifth 3-way valve 550 has a handle-type lever 554 in the middle of a T-shaped body. According to the rotational direction of the lever 554, two flow channels are selectively formed; one from the first port 551 to the second port 552 and the other from the first port 551 to the third port 553.

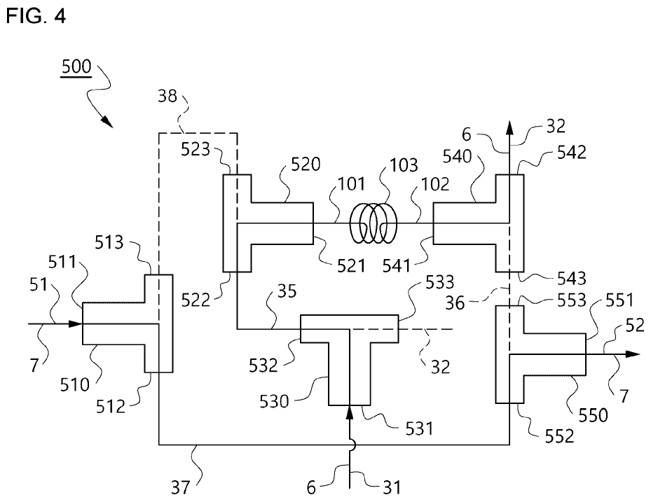

Hereinafter, the operation of the valve assembly 500 will be described in detail with reference to FIGS. 4 to 11.

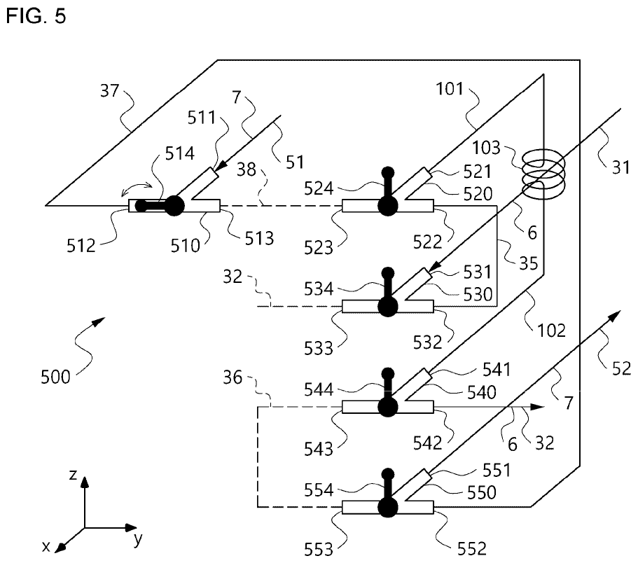

FIGS. 4 and 5 show the valve assembly 500 in the sampling connection operation. FIG. 4 is a schematic diagram of the valve assembly 500, and FIG. 5 is a schematic diagram showing the arrangement of each valve of the valve assembly 500 in three dimensions.

While in the sampling connection operation, in the first 3-way valve 530, the first port 531 and the second port 532 are in communication with each other to form the flow channel from the first port 531 to the second port 532. In this instance, the flow channel to the third port 533 is interrupted.

In the second 3-way valve 520, the first port 521 and the second port 522 are in communication with each other to form the flow channel from the first port 521 to the second port 522. In this instance, the flow channel to the third port 523 is interrupted.

In the third 3-way valve 540, the first port 541 and the second port 542 are in communication with each other to form the flow channel from the first port 541 to the second port 542. In this instance, the flow channel to the third port 543 is interrupted.

Accordingly, parts of the gas mixture 6 flowing in the pipe 20 go into the first port 531 of the first 3-way valve 530 through the upstream connection pipe 31 and enter the sampling unit 11 through the second 3-way valve 520. The gas mixture 6 leaves the sampling unit 11, flows to the third 3-way valve 540 and returns to the pipe 20 through the downstream connection pipe 32.

During the sampling connection operation, the gas mixture 6 continuously flows in the sampling unit 11, and thus the sampling unit 11 is always filled with the same volume of gas mixture 6.

On the other hand, while in the sampling connection operation, in the fourth 3-way valve 510, the first port 511 and the second port 512 are in communication with each other to form the flow channel from the first port 511 to the second port 512. In this instance, the flow channel to the third port 513 is interrupted.

In the fifth 3-way valve 550, the first port 551 and the second port 552 are in communication with each other to form the flow channel from the first port 551 to the second port 552. In this instance, the flow channel to the third port 553 is interrupted.

Accordingly, the channel of fluid flow to the carrier gas inlet pipe 51, the fourth 3-way valve 510, the fifth 3-way valve 550 and the detection unit inlet pipe 52 is formed, thereby forming the channel of fluid flow from the carrier gas tank 70 directly to the detection unit 12.

As described above, through the channel of fluid flow, the carrier gas 7 continuously flows from the carrier gas tank 70 toward the detection unit 12, and thus a clean atmosphere in which the detection unit 12 is not contaminated by the outdoor air is maintained by the carrier gas 7.

In the sampling connection operation state, when the levers of the first to fifth 3-way valves are selectively turned by a link structure 600, each 3-way valve operates to switch the fluid channel, so it is changed to the sampling interruption operation state.

According to this embodiment, in the sampling interruption operation step, the two-step operation of a sampling route closing operation and a depressurization operation is performed in a sequential order.

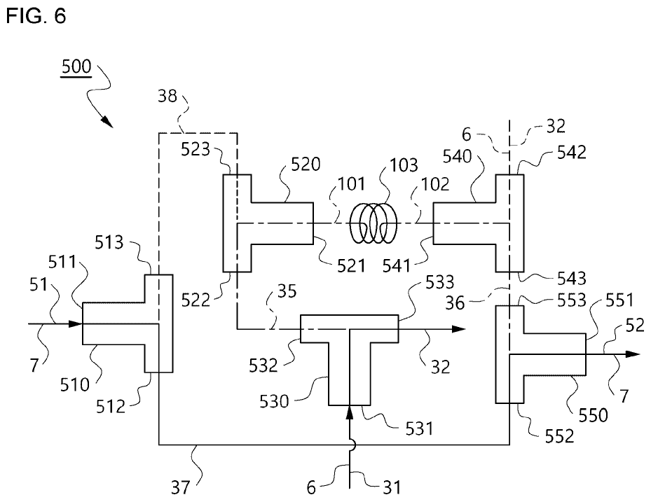

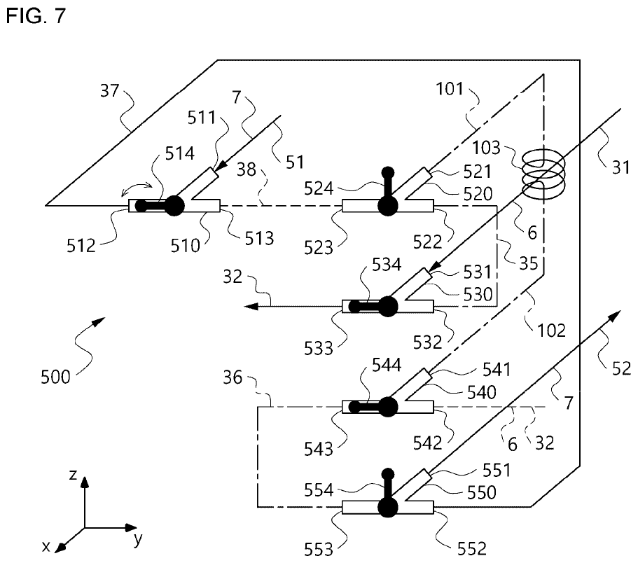

FIGS. 6 and 7 show the valve assembly 500 in the sampling route closing operation. FIG. 6 is a schematic diagram of the valve assembly 500, and FIG. 7 is a schematic diagram showing the arrangement of each valve of the valve assembly 500 in three dimensions.

According to this embodiment, when it is changed to the sampling interruption operation, first, the sampling route closing operation is performed. The sampling route closing operation is performed to trap the gas mixture 6 in the sampling unit.

In the sampling route closing operation, the first 3-way valve 530 operates to block the second port 532 and bring the first port 531 and the third port 533 into communication with each other to form the flow channel from the first port 531 to the third port 533.

Additionally, at the same time as the operation of the first 3-way valve 530, the third 3-way valve 540 operates to block the second port 542 and bring the first port 541 and the third port 543 into communication with each other to form the flow channel from the first port 541 to the third port 543.

Accordingly, the communication between the sampling unit 11 and the pipe 20 is interrupted, and the gas mixture 6 is trapped in the sampling unit 11. Specifically, the gas mixture 6 is trapped between the first 3-way valve 530 and the second 3-way valve 520 (the first connection pipe 35), between the second 3-way valve 520 and the third 3-way valve 540 (a sampler module 103) and between the third 3-way valve 540 and the fifth 3-way valve 550 (the third connection pipe 36). In FIGS. 6 and 7, the alternating long and short dashed line indicates where the fluid is trapped and does not flow.

On the other hand, the gas mixture 6 entering the upstream connection pipe 31 returns to the pipe 20 through the third port 533 of the first 3-way valve 530.

Additionally, the flow channel of the carrier gas leading to the carrier gas inlet pipe 51, the fourth 3-way valve 510, the fifth 3-way valve 550 and the detection unit inlet pipe 52 is maintained. The carrier gas 7 continuously flows from the carrier gas tank 70 toward the detection unit 12, and thus a clean atmosphere in which the detection unit 12 is not contaminated by the outdoor air is maintained by the carrier gas 7.

After the sampling route closing operation is performed, the depressurization operation is performed. The depressurization operation is performed to reduce the pressure of the gas mixture 6 trapped in the sampling unit 11.

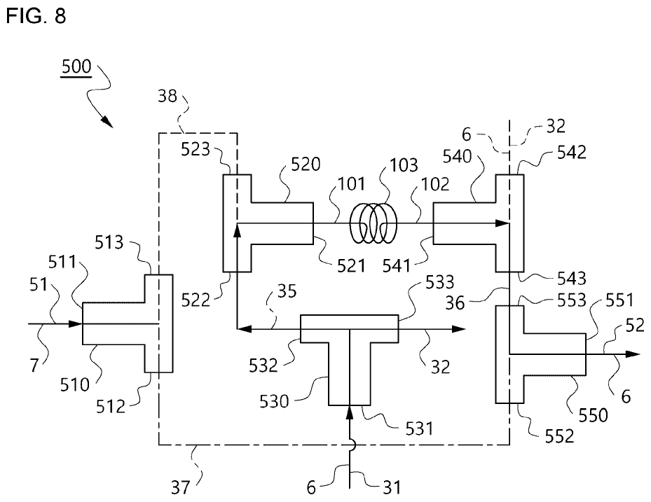

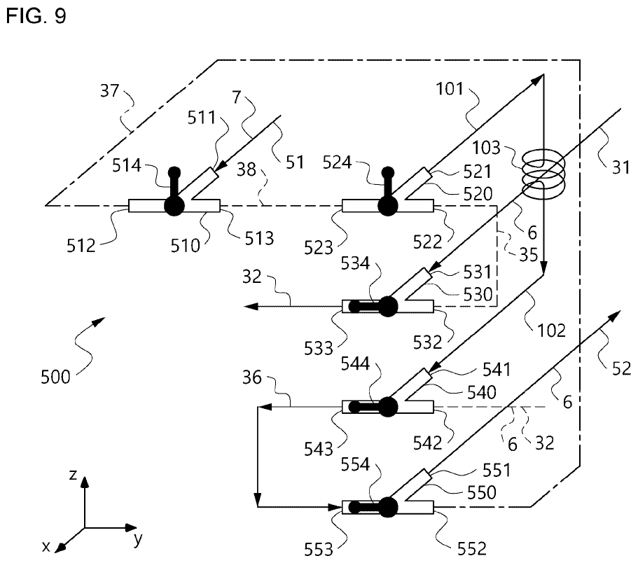

FIGS. 8 and 9 show the valve assembly 500 in the depressurization operation. FIG. 8 is a schematic diagram of the valve assembly 500, and FIG. 9 is a schematic diagram showing the arrangement of each valve of the valve assembly 500 in three dimensions.

In the depressurization operation, the fifth 3-way valve 550 operates to block the second port 552 and bring the first port 551 and the third port 553 to communication with each other to form the flow channel from the first port 551 to the third port 553.

Accordingly, the carrier gas flow channel leading to the sampling unit 11 is interrupted, and the carrier gas 7 does not enter the sampling unit 11. However, the carrier gas 7 is trapped and kept in the fourth connection pipe 37 inside the sampling unit 11. Accordingly, when it is changed to the gas delivery operation as described below, the flow of the carrier gas 7 may immediately occur. In FIGS. 8 and 9, the alternating long and short dashed line indicates where the fluid is trapped and does not flow.

On the other hand, the gas mixture 6 trapped in the sampling unit 11 flows to the detection unit inlet pipe 52 by a pressure difference and enters the depressurization chamber 600.

The depressurization chamber 600 is a chamber having a larger diameter than the detection unit inlet pipe 52. As the volume of the space increases, the pressure of the gas mixture 6 is reduced in the depressurization chamber 600. For example, the pressure of the gas mixture 6 having the pressure of 5 bar is reduced to 2 bar in the depressurization chamber 600.

According to this embodiment, as shown in FIG. 1, to prevent condensation of the gas mixture 6 in the depressurization chamber 600 as the pressure is reduced, a heater 601 is used to heat the depressurization chamber 600 to a predetermined temperature. The heater 601 maintains the depressurization chamber 600 at or above the predetermined temperature (for example, target temperature 100° C. or more) to prevent adsorption of the gas mixture 6 onto the chamber. The depressurization chamber 600 may be, for example, made of stainless steel.

In the high pressure environment (for example, 5 bar or more) of the flowing gas mixture 6, when the gas mixture 6 trapped in the sampling unit 11 is allowed to flow into the detection unit 12, the internal components of the detection unit 12 may be damaged by the pressure.

The pressure of the gas mixture 6 may be reduced to a measurable level by the internal components of the detection unit 12 through the depressurization operation of reducing the pressure of the gas mixture 6.

After the sampling interruption operation is performed, the valve assembly 500 operates to perform the gas delivery operation to form the channel of fluid flow from the carrier gas tank 70 to the detection unit through the sampling unit 11.

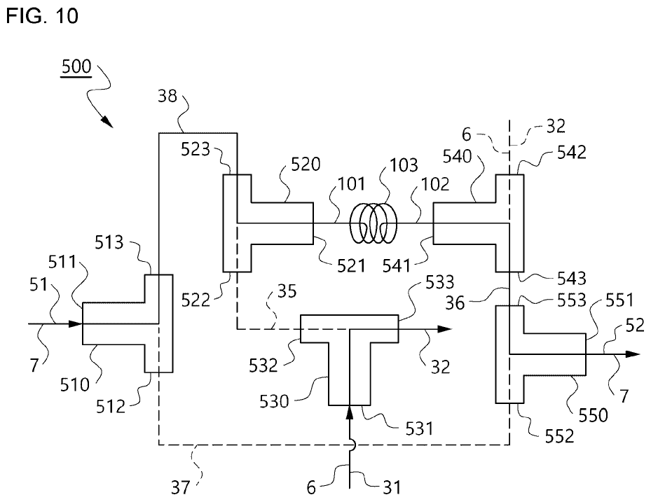

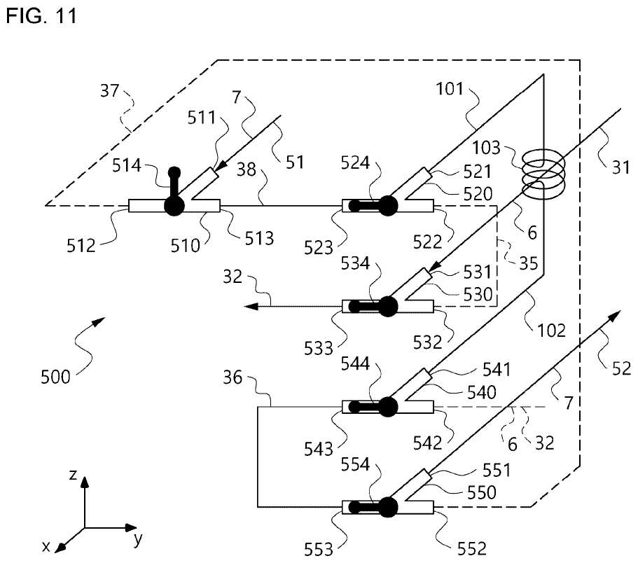

FIGS. 10 and 11 show the valve assembly 500 in the gas delivery operation. FIG. 10 is a schematic diagram of the valve assembly 500, and FIG. 11 is a schematic diagram showing the arrangement of each valve of the valve assembly 500 in three dimensions.

In the gas delivery operation, the second 3-way valve 520 operates to block the second port 522 and bring the first port 521 and the third port 523 into communication with each other to form the flow channel from the first port 521 to the third port 523.

Additionally, at the same time as the operation of the second 3-way valve 520, the fourth 3-way valve 510 operates to block the second port 512 and bring the first port 511 and the third port 513 into communication with each other to form the flow channel from the first port 511 to the third port 513.

In the gas delivery operation, the second 3-way valve 520 and the fourth 3-way valve 510 are in communication with each other to form the channel of fluid flow from the carrier gas tank 70 to the detection unit 12 through the sampling unit 11. That is, according to this embodiment, the gas delivery operation is performed to form the channel of fluid flow from the carrier gas tank 70 to the detection unit 12 through the sampling unit 11. The gas mixture 6 entering the upstream connection pipe 31 returns to the pipe 20 through the third port 533 of the first 3-way valve 530.

The carrier gas 7 provides forces not only to push out the gas mixture 6 remaining in the sampling unit 11 but also to allow the gas mixture 6 to flow in the detection unit 12.

In this embodiment, the valve assembly 500 operates to perform four steps of the sampling connection operation, the sampling interruption operation including the sampling route closing operation and the depressurization operation and the gas delivery operation in a sequential order. However, the time required to perform each operation is so short that there are almost no time differences. Additionally, before the intended operation in each operation step is completely performed, the operation of the next step may be performed. For example, even though the gas mixture 6 did not completely leave the sampling unit 11, when the pressure of the gas mixture 6 is reduced to the target pressure by the depressurization chamber 600 through the depressurization operation of reducing the pressure, it may be immediately changed to the gas delivery operation.

According to this embodiment, as the valve assembly 500 includes the plurality of valves, it is possible to relatively freely arrange the plurality of valves, thereby easily installing in the existing treatment apparatus 1. Additionally, it is easy to replace faulty valves, thereby reducing the repair and maintenance efforts and costs.

Furthermore, according to this embodiment, because the multi-port valves are applied as the plurality of valves, it may be possible to suppress unnecessary valve utilization as much as possible.

Moreover, according to this embodiment, the manual 3-way valve that is operated by the lever is applied as the valve. In general, the pressure threshold the manual 3-way valve can withstand is higher than an automatic 6-way valve. Accordingly, the measuring system 10 according to this embodiment has higher pressure resistance than a measuring system using one automatic 6-way valve, and can be used in applications of very high pressure acting on the pipe 20.

Additionally, as the sampling route is closed and depressurization is performed, followed by gas delivery to conduct an analysis, the measuring system can effectively operate in a high pressure environment.

### Variation

In Embodiment 1, to reduce the pressure of the gas mixture 6, the operation of four steps is performed by selectively manipulating (operating) the levers of the first to fifth 3-way valves. However, in an environment in which it is not necessary to reduce the pressure of the gas mixture 6, the present disclosure is not limited thereto.

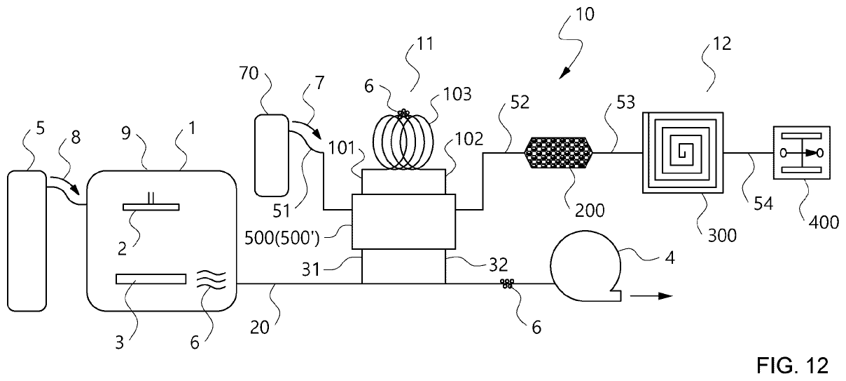

In the environment in which it is not necessary to reduce the pressure of the gas mixture 6, the valve operation in the valve assembly 500 shown in FIG. 3 may be performed in a different way from Embodiment 1 to simplify the operation steps. Additionally, as shown in FIG. 12, in the environment in which it is not necessary to reduce the pressure of the gas mixture 6, the depressurization chamber 600 and the heater 601 may be also omitted. FIG. 12 is the same as FIG. 1 except that the illustration of the depressurization chamber 600 and the heater 601 is omitted, and redundant descriptions are omitted.

Hereinafter, the operation of the valve assembly 500 according to the variation with the simplified operation steps will be described with reference to FIGS. 4, 5, 10 and 11. In other words, in this variation, the operation of two steps including the operation of FIG. 4 (FIG. 5) and the operation of FIG. 10 (FIG. 11) is performed, and the operation corresponding to FIGS. 6 to 9 is omitted.

According to the variation, when the sampling connection operation is changed to the sampling interruption operation, at the same time as the sampling interruption operation, the valve assembly 500 is switched to the gas delivery operation to form the channel of fluid flow from the carrier gas tank 70 to the detection unit through the sampling unit 11.

Overlapping descriptions with the sampling connection operation described with reference to FIGS. 4 and 5 are omitted.

According to the variation, in the sampling connection operation state, when the levers of the first to fifth 3-way valves are simultaneously turned, each 3-way valve simultaneously operates to simultaneously switch the fluid channels, so it is changed to the sampling interruption operation state, and at the same time, it is also changed to the gas delivery operation state.

Referring to FIGS. 10 and 11, in the sampling interruption operation (the gas delivery operation), the first 3-way valve 530 operates to block the second port 532 and bring the first port 531 and the third port 533 into communication with each other to form the flow channel from the first port 531 to the third port 533.

Additionally, at the same time as the operation of the first 3-way valve 530, the second 3-way valve 520 operates to block the second port 522 and bring the first port 521 and the third port 523 into communication with each other to form the flow channel from the first port 521 to the third port 523.

Additionally, at the same time as the operation of the first 3-way valve 530, the third 3-way valve 540 operates to block the second port 542 and bring the first port 541 and the third port 543 into communication with each other to form the flow channel from the first port 541 to the third port 543.

Additionally, at the same time as the operation of the first 3-way valve 530, the fourth 3-way valve 510 operates to block the second port 512 and bring the first port 511 and the third port 513 into communication with each other to form the flow channel from the first port 511 to the third port 513.

Additionally, at the same time as the operation of the first 3-way valve 530, the fifth 3-way valve 550 operates to block the second port 552 and bring the first port 551 and the third port 553 into communication with each other to form the flow channel from the first port 551 to the third port 553.

Accordingly, the communication between the sampling unit 11 and the pipe 20 is interrupted, and the gas mixture 6 is trapped in the sampling unit 11.

In this instance, according to this embodiment, in the sampling interruption operation, the second 3-way valve 520 and the fourth 3-way valve 510 are in communication with each other and the third 3-way valve 540 and the fifth 3-way valve 550 are in communication with each other, and thus at the same time as the sampling interruption operation, the channel of fluid flow from the carrier gas tank 70 to the detection unit 12 through the sampling unit 11 is formed. That is, according to this embodiment, the gas delivery operation of forming the channel of fluid flow from the carrier gas tank 70 to the detection unit 12 through the sampling unit 11 is performed at the same time as the sampling interruption operation.

The gas mixture 6 trapped in the sampling unit 11 is pushed towards the detection unit 12 by the carrier gas 7 from the carrier gas tank 70.

On the other hand, the gas mixture 6 entering the upstream connection pipe 31 returns to the pipe 20 through the third port 533 of the first 3-way valve 530.

According to the variation, in the sampling connection operation state, when the levers of the first to fifth 3-way valves are simultaneously turned, each 3-way valve simultaneously operates to simultaneously switch the fluid channels, so it is changed to the sampling interruption operation state.

To simultaneously operate the plurality of 3-way valves, a means for electrical control may be contemplated, but according to this variation, each 3-way valve is simultaneously operated by the operation of the mechanical link structure 700.

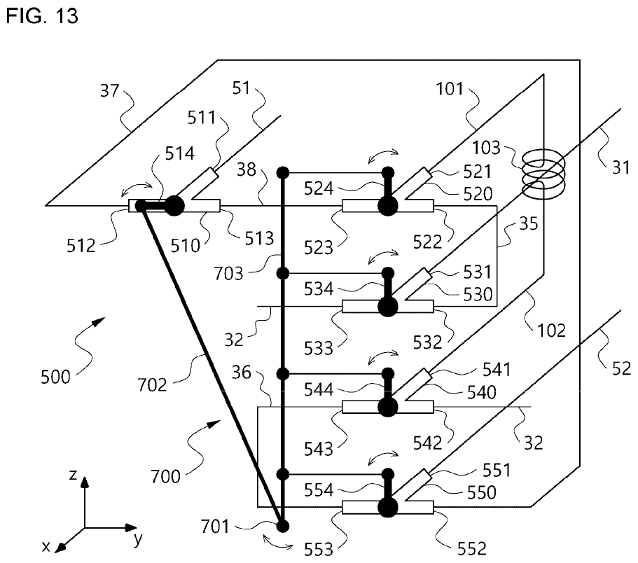

FIG. 13 is a schematic diagram of the valve assembly 500 according to the variation.

As shown in FIG. 13, according to this embodiment, the lever of each of the 3-way valves is connected to one link structure 700 together. The link structure 700 includes link arms 702, 703 that rotate around a motor 701, and the link arms 702, 703 are rotatably connected to the lever of each of the 3-way valves. According to this embodiment, when the link arms 702, 703 are rotated by the rotation of the motor 701, the lever of each of the 3-way valves connected with the link arms simultaneously rotates to simultaneously switch the channel of fluid flow in each valve.

It should be understood that FIG. 3 shows the schematic structure of the link structure 700 to describe the concept of the link structure 700. The link structure 700 is not limited to a particular one and may include those configured to simultaneously turn the lever of each of the 3-way valves by a single driving means (a motor, etc.). Additionally, the link structure 700 is not limited to the rotatably connected link arm structure, and may be formed using a combination of known driving means such as a rack and pinion.

According to the variation, each 3-way valve is simultaneously operated by the operation of the link structure 700, thereby matching the timing of operation of each valve all at once without a complex and elaborate control algorithm or control means. Accordingly, it may be possible to make it easy to control the entire system and reduce the cost.

However, in the same way as Embodiment 1, the manual 3-way valve that is operated by the lever is applied as the valve, and thus the pressure threshold the manual 3-way valve can withstand is high. Accordingly, in this variation, the measuring system can be also used in applications of relatively high pressure acting on the pipe 20.

### Embodiment 2

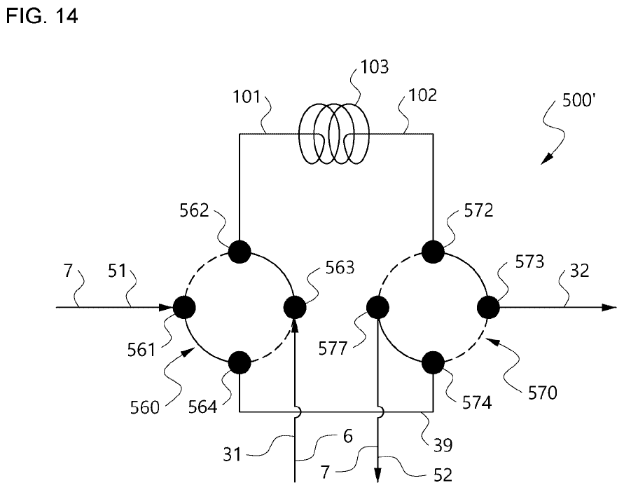

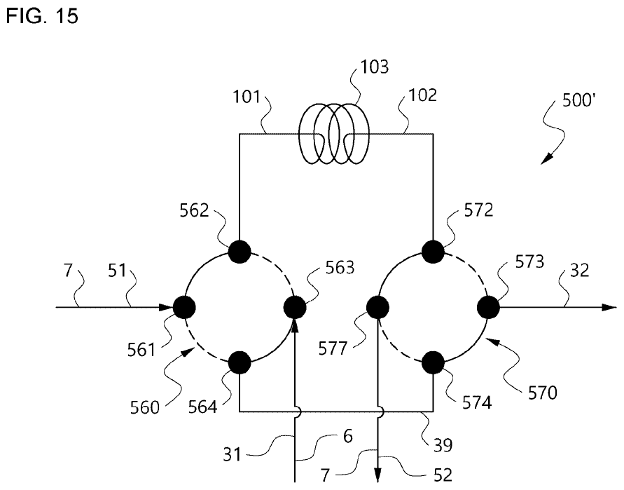

FIGS. 14 and 15 are schematic diagrams of the valve assembly 500′ according to another embodiment.

As shown in FIGS. 14 and 15, the valve assembly 500′ according to this embodiment includes multi-port valves, i.e., two 4-way valves 560, 570, each having four ports. According to this embodiment, the two 4-way valves 560, 570 are an “automatic valve” that automatically operates by the control of a controller (not shown).

A first port 561 of the first 4-way valve 560 is connected to the carrier gas inlet pipe 51 that communicates with the carrier gas tank 70, a second port 562 is connected to the entrance 101 of the sampling unit 11, a third port 563 is connected to the upstream connection pipe 31 and connected to the pipe 20, and a fourth port 564 is connected to an end of a fifth connection pipe 39.

A first port 571 of the second 4-way valve 570 is connected to the detection unit inlet pipe 52 that communicates with the detection unit 12, a second port 572 is connected to the exit 102 of the sampling unit 11, a third port 573 is connected to the downstream connection pipe 32 and connected to the pipe 20, and a fourth port 574 is connected to an opposite end of the fifth connection pipe 39.

As shown in FIG. 14, while in the sampling connection operation, in the first 4-way valve 560, the second port 562 and the third port 563 are in communication with each other, and the first port 561 and the fourth port 564 are in communication with each other. Likewise, in the second 4-way valve 570, the second port 572 and the third port 573 are in communication with each other and the first port 571 and the fourth port 574 are in communication with each other.

Accordingly, parts of the gas mixture 6 flowing in the pipe 20 go into the third port 563 of the first 4-way valve 560 through the upstream connection pipe 31, and enter the sampling unit 11 through the second port 562. The gas mixture 6 leaves the sampling unit 11, flows into the second port 572 of the second 4-way valve 570, and returns to the pipe 20 via the downstream connection pipe 32 through the third port 573.

During the sampling connection operation, the gas mixture 6 continuously flows in the sampling unit 11, and thus the sampling unit 11 is always filled with the same volume of gas mixture 6.

On the other hand, in the sampling connection operation, the channel of fluid flow to the carrier gas inlet pipe 51, the fourth 3-way valve 510, the fifth 3-way valve 550 and the detection unit inlet pipe 52 is formed, thereby forming the channel of fluid flow from the carrier gas tank 70 directly to the detection unit 12.

As described above, through the channel of fluid flow, the carrier gas 7 continuously flows from the carrier gas tank 70 toward the detection unit 12, and thus a clean atmosphere in which the detection unit 12 is not contaminated by the outdoor air is maintained by the carrier gas 7.

In the sampling connection operation state, the first 4-way valve 560 and the second 4-way valve 570 are simultaneously operated by the controller (not shown) to switch the fluid channel, so it is changed to the sampling interruption operation state.

As shown in FIG. 15, in the sampling interruption operation, the first 4-way valve 560 operates to bring the third port 563 and the fourth port 564 into communication with each other and interrupt the communication between the second port 562 and the third port 563, and at the same time, the second 4-way valve 570 operates to bring the third port 573 and the fourth port 574 into communication with each other and interrupt the communication between the second port 562 and the third port 563. Accordingly, the communication between the sampling unit 11 and the pipe 20 is interrupted, and the gas mixture 6 is trapped in the sampling unit 11.

Additionally, after or simultaneously with the sampling interruption operation, the controller of the valve assembly 500′ brings the first port 661 and the second port 562 of the first 4-way valve 560 into communication with each other and brings the first port 671 and the second port 572 into communication with each other.

Accordingly, the channel of fluid flow from the carrier gas tank 70 to the detection unit 12 through the sampling unit 11 is formed to perform the gas delivery operation. The gas mixture 6 trapped in the sampling unit 11 is pushed towards the detection unit 12 by the carrier gas 7 from the carrier gas tank 70.

On the other hand, the gas mixture 6 entering the upstream connection pipe 31 returns to the pipe 20 through the third port 563 and the fourth port 564 of the first 4-way valve 560 and the fourth port 574 and the third port 573 of the second 4-way valve 570.

In this embodiment 2, in the same way as the variation of Embodiment 1, although it has been described that the valve assembly 500′ has the operation steps of the sampling connection operation and the sampling interruption operation (and the gas delivery operation), it will be understood that the valve assembly 500′ may perform the operation steps of the sampling connection operation, the sampling route closing operation, the depressurization operation and the gas delivery operation.

According to this embodiment, as the valve assembly 500′ includes the plurality of valves, it is possible to relatively freely arrange the plurality of valves, thereby easily installing in the existing treatment apparatus 1. Additionally, it is easy to replace faulty valves, thereby reducing the repair and maintenance efforts and costs. Furthermore, according to this embodiment, because the multi-port valves are applied as the plurality of valves, it may be possible to suppress unnecessary valve utilization as much as possible. Although the 3-way valve or the 4-way valve is described as the multi-port valve in this embodiment, it will be understood that the so-call “multi-port valve” having the plurality of ports to switch the fluid flow can exert the similar effect by its combination.

Moreover, according to this embodiment, the 4-way valve that is operated by the lever is applied as the valve. In general, the pressure threshold the 4-way valve can withstand is higher than a 6-way valve. Accordingly, the measuring system 10 according to this embodiment has higher pressure resistance than a measuring system using a 6-way valve, and can be used in applications of very high pressure acting on the pipe 20.

### Detection Unit 12

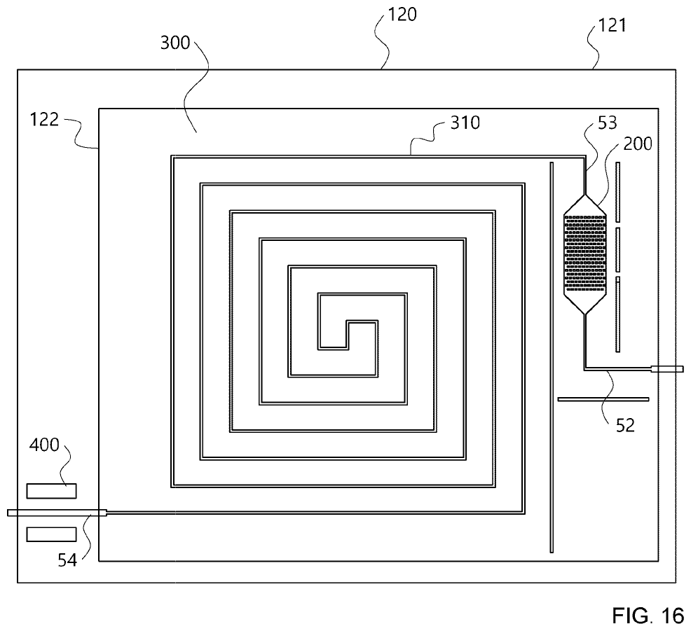

Hereinafter, the configuration of the detection unit 12 and the principle of separation and detection of the gaseous phase materials 60 included in the gas mixture 6 by the detection unit 12 will be described with reference to FIGS. 16 to 21.

According to this embodiment, the concentration module 200, the separation module 300 and the sensor module 400 of the detection unit 12 are incorporated and integrated into a portable compact detection device 120. As described above, the depressurization chamber 600 and the heater 601 may be omitted depending on the pressure environment, and in the following description of the detection unit 12, a detailed description of the depressurization chamber 600 and the heater 601 is omitted.

FIG. 16 is a schematic conceptual diagram of the detection device 120.

As shown in FIG. 16, the detection device 120 has a structure in which a substrate 122 on which the concentration module 200 and the separation module 300 are integrated and the sensor module 400 are installed in a single body 121. A heat line, a power source (not shown) of the sensor module 400 and a communication device (not shown) for transmission of a signal of the sensor module 400 are embedded in the body 121. Additionally, the body 121 may include a memory to store information such as libraries associated with the time taken by each gaseous phase material 60 to exit a separation path 310 as described below, and a processor to derive the results by comparing information detected by the sensor module with the libraries. Furthermore, the body 121 may include a speaker or a liquid crystal display to notify the derived results to a worker. However, the functions of the memory and the processor may be performed by another computer, and the detection device 120 may transmit the detection results by the sensor module to the computer via communication with another computer.

The substrate 122 is formed by bonding a plate (a first substrate) of silicone and a plate (a second substrate) of glass.

According to this embodiment, the concentration module 200, the separation module 200 and the plurality of pipelines 52, 53 connecting them are formed on one surface of the first substrate by deep etching using a deep reactive-ion etching (DRIE) process. Accordingly, it is possible to elaborately form a nano size structure on the substrate 122, thereby minimizing the total size of the detection device 120. The concentration module 200, the separation module 300 and the plurality of pipelines 52, 53 connecting them are simultaneously formed on the first substrate into a concave groove shape by etching, followed by placing and bonding the second substrate, thus completing a structure having the closed top with the concave groove closed.

According to this embodiment, the first substrate and the second substrate may be strongly bonded to each other in an atmospheric condition by anodic bonding that is a voltage assisted bonding process.

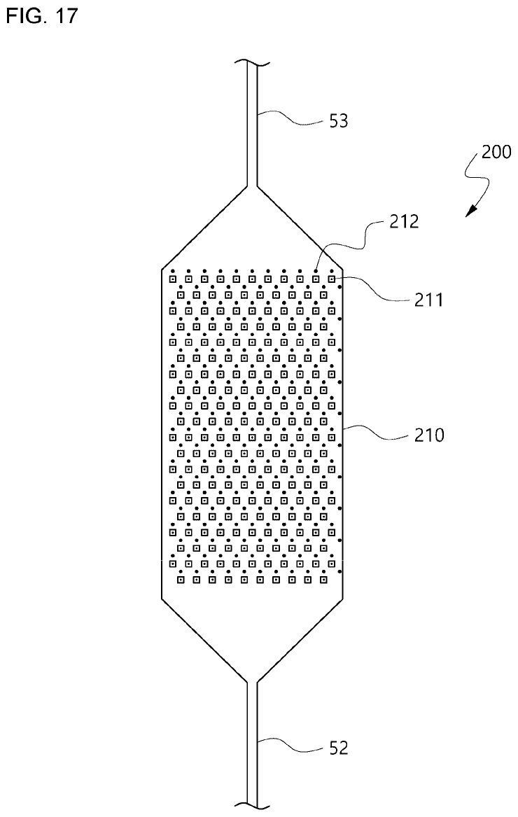

FIG. 17 is an enlarged diagram of the concentration module 200 according to this embodiment.

The concentration module 200 includes a concentration chamber 210 which is a space of larger volume than the pipeline connected to the concentration module 200.

The concentration chamber 210 includes two facing short sides extended in the short side direction of the concentration chamber 210, and two facing long sides extended in the long side direction of the concentration chamber 210, and has an approximately long polygonal shape.

The short side is bent in an approximately “v” shape with the center facing away from the long side, thereby achieving uniform distribution of the fluid flowing in the concentration chamber 210.

The entry pipeline 52 in communication with the concentration chamber 210 to allow the gas mixture 6 to enter is formed at the center of one short side of the concentration chamber 210. The exit pipeline 53 through which gas exits the concentration chamber 210 is formed at the center of the other short side of the concentration chamber 210.

The “entry” and “exit” as used herein are intended to refer to different openings through which the fluid enters and exits the corresponding pipeline, and are not necessarily intended to define that the fluid enters at the entry and exits at the exit in the corresponding pipeline. That is, in some cases, the fluid may enter at the exit and exit at the entry in the corresponding pipeline.

A plurality of pillars 211 is arranged at a predetermined interval in the concentration chamber 210. When DRIE is used, the plurality of pillars 211 may be formed in the concentration chamber 210 by leaving parts of the concentration chamber 210 unetched when forming the concentration chamber 210.

The concentration chamber 210 extracts the gaseous phase materials 60 in the gas mixture 6, and concentrates and stores the gaseous phase materials 60. To this end, the concentration chamber 210 is filled with an adsorbent 212 to trap the gaseous phase materials 60. The adsorbent 212 may include, for example, materials such as carbon compounds, to which the gaseous phase materials 60 which are organic compounds attach by van der Waals forces, to trap the gaseous phase materials 60.

The adsorbent 212 may be pre-filled in the chamber 110 before bonding the first substrate and the second substrate, and the adsorbent 212 may be filled in the concentration chamber 210 by a gas transfer method. In the case of the gas transfer method, an inlet pipe (not shown) in communication with the concentration chamber 210 may be formed.

The gaseous phase materials 60 entering the concentration chamber 210 is trapped on the adsorbent 212 and concentrated and stored in the concentration chamber 210.

To discharge the gaseous phase materials 60 concentrated and stored in the concentration chamber 210, it is necessary to break the bonds between the adsorbent 212 and the gaseous phase materials 60, and the detection device 120 according to this embodiment includes a heating device to heat the concentration chamber 210.

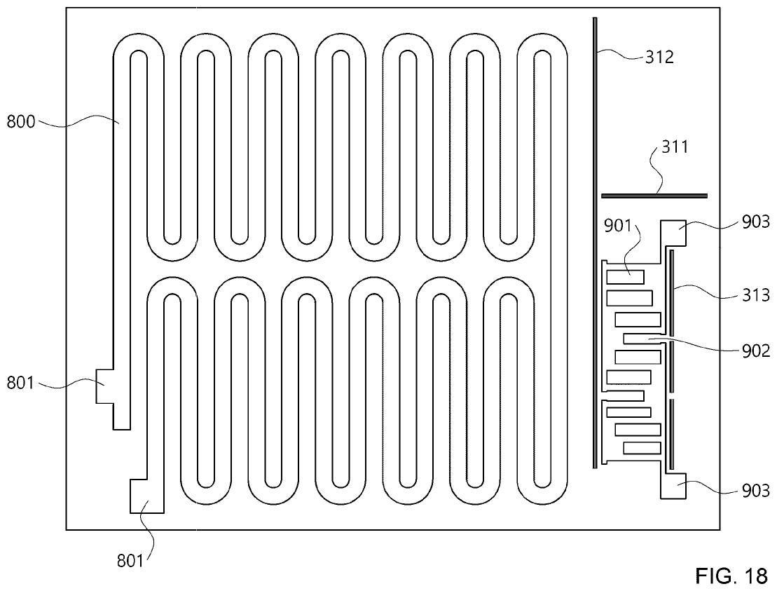

FIG. 18 shows the rear surface of the first substrate.

The heat line 901 as the chamber heating device is attached to the rear surface of the first substrate to generate heat when the power is applied. The heat line 901 is formed in the first substrate at a location corresponding to the concentration chamber 210. The heat line 901 has a terminal 903 for connection to the power source. A temperature sensor 902 may be disposed at the center of the heat line 901 to measure the temperature that increases by the heat line 901.

When heat is generated by applying the power to the heat line 901, thermal energy that may debond the adsorbent 212 and the gaseous phase materials 60 may be selectively applied to the concentration chamber 210.

Meanwhile, as shown in FIG. 18, according to this embodiment, a heat line 800 that selectively applies heat to the separation path 310 to improve the reactivity in the separation path 310 as described below may be formed in the rear surface of the first substrate at a location corresponding to the separation path 310. The heat line 800 has a terminal 801 for applying the power at two ends.

In this instance, the heat applied by the heat line 901 may be unexpectedly applied to the adjacent component such as the separation path 310 by thermal conduction by the first substrate of silicon.

To prevent the thermal conduction as much as possible, according to this embodiment, a plurality of slits 311, 312, 313 is formed around the heat line 901 and completely passes through the first substrate.

The pipeline 53 in communication with the concentration module 200 is in communication with the separation module 300.

The separation module 300 includes the elongated separation path 310. The separation path 310 forms a single fluid flow path, and the gaseous phase materials 60 entering the separation path 310 are separated into substances while moving along the separation path 310 having the very long path and discharged from the separation path 310 at time intervals.

According to this embodiment, for the separation path 310 to have a long path enough to separate hazardous materials, the separation path 310 is disposed to form a single layer of column bent in a maze pattern within a set rectangular or square space.

As shown in FIG. 16, the separation path 310 is bent from the entry of the separation path 310 in fluid communication with the conduit 53 to the center of the rectangular or square space, extended in the shape of a sort of coil, and then extended from the center to the exit of the separation path 310 in a coil shape. That is, the path in the column form extended in a serpentine shape toward the center and the path extended facing away from the center are arranged adjacent to each other in an alternating manner, thereby maximizing the path length of the separation path 310 within the small space.

Although FIG. 16 shows the adjacent paths spaced apart at sufficient intervals for convenience of illustration, the interval between the adjacent paths is very short.

Since the paths are arranged at very short intervals, the separation path 310 having a cross sectional area of a few nanometer level may be, for example, extended over about 3 m.

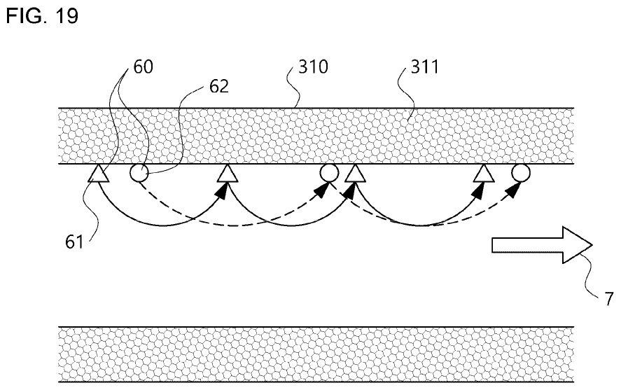

FIG. 19 schematically shows the inside of the separation path 310 according to an embodiment of the present disclosure.

As shown in FIG. 19, the inner surface of the separation path 310 is coated with a porous material 311 to which the gaseous phase materials 60 may attach. For example, the porous material 311 may be a porous polymer such as PDMS.

The hazardous materials M which are organic compounds attach to the porous polymer by van der Waals forces. In this instance, when the carrier gas 7 flows in the separation path 310, the gaseous phase materials 60 attached to the porous material 311 separate from the porous material 311 and flow a predetermined distance by the force of the carrier gas 7, and then lose the mobility and attach to the porous material 311 again, and this process repeats.

Since the gaseous phase materials 60 differ in mass and van der Waals forces acting between the gaseous phase materials 60 and the porous material 311 depending on the substances, the gaseous phase materials 60 of different substances attach to the porous material 311, separate from the porous material 311 and then flow at different frequencies and distances as shown in FIG. 19. That is, the gaseous phase materials 60 move at different movement speeds in the separation path 310 for each substance. For example, the second material 62 indicated by a circle moves faster than the first material 61 indicated by a triangle.

According to this embodiment, since the separation path 310 has the long path amounting to about 3 m, the movement distance is equalized for each substance while the gaseous phase materials 60 injected through the entry of the separation path 310 are moving along the long path, and the gaseous phase materials 60 come out at the exit of the separation path 310 for each substance. Since the gaseous phase materials 60 have different movement speeds depending on the substances, the gaseous phase materials 60 are separated into substances and come out at the exit of the separation path 310 at time intervals. That is, only by traveling the hazardous materials through the separation path 310 without applying electricity, the gaseous phase materials 60 are separated into substances and discharged at time intervals.

The porous material 311 may be coated on the separation path 310 before the first substrate and the second substrate are bonded to each other, and may be coated by a gas flow method through the inlet pipe (not shown).

The gaseous phase materials 60 leaving the exit of the separation path 310 in a sequential order are detected by the sensor module 400.

The sensor module 400 according to this embodiment is a photoionization detection (PID) sensor that measures a voltage change by electrons released from the gaseous phase materials 60 by applying UV to the gaseous phase materials 60 from the separation path 310 of the separation module 300. Specifically, when a material such as an organic compound is illuminated with UV, a potential occurs due to electron emission.

As the concentration of the corresponding material is higher, the detected potential value is higher, and through this, the concentration of the corresponding material may be calculated.

The sampled gas mixture 6 is pushed by the carrier gas 7 into the concentration chamber 210 through the detection unit inlet 52. The gas entering the concentration chamber 210 moves in the long side direction of the concentration chamber 210. In this process, the gaseous phase materials 60 included in the gas mixture 6 are adsorbed onto the adsorbent 212 filled in the concentration chamber 210.

The gaseous phase materials 60 are concentrated in the concentration chamber 210 for the predetermined time, and the concentration chamber 210 is heated by applying the power to the heat line 500. The gaseous phase materials 60 concentrated and stored in the concentration chamber 210 are separated from the adsorbent 212 by the applied thermal energy, and the carrier gas 7 flowing through the concentration chamber 210 carries the gaseous phase materials 60 out of the concentration chamber 210. The carrier gas 7 carrying the gaseous phase materials 60 flows to the separation path 310.

The gaseous phase materials 60 of high concentration from the concentration chamber 210 are instantaneously fed into the separation path 310.

That is, the concentration chamber 210 according to this embodiment acts as not only a reservoir to concentrate and store the hazardous materials, but also an injector to inject the high concentration hazardous materials into the separation path 310.

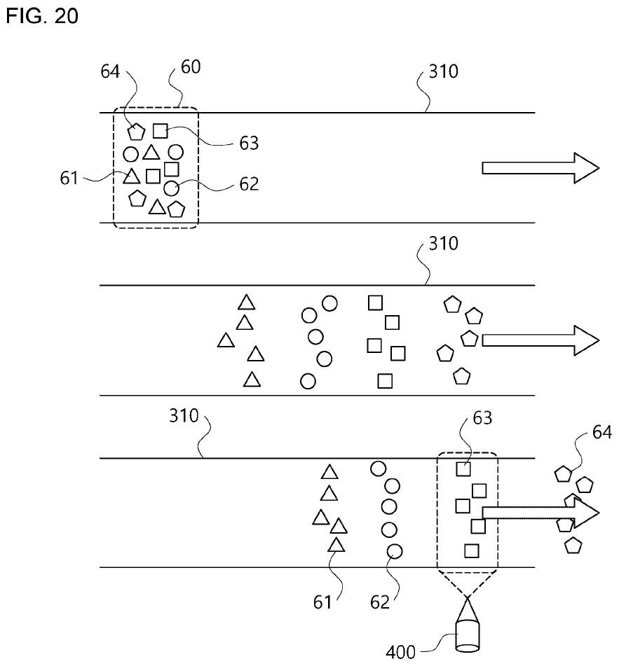

FIG. 20 schematically shows the process of separating and detecting the gaseous phase materials 60 using the detection device 120 according to this embodiment.

As described above, the gaseous phase materials 60 have different movement speeds in the separation path 310 depending on the substances.

Through experiments, the time taken by the gaseous phase materials 60 to exit the separation path 310 for each substance may be pre-acquired.

For example, in the case of gas containing isopropylantipyrine (IPA) alone, the corresponding material may be detected by the sensor module 400 in about 20 seconds. In this way, the experiments may be performed on each of the expected gaseous phase materials 60 to generate the libraries of the time taken by each gaseous phase material 60 to exit the separation path 310.

Since the gaseous phase materials 61, 62, 63, 64 of different substances are discharged through the separation path 310 in a sequential order, the substances of the corresponding gaseous phase materials may be identified by identifying the time at which the potential value remarkably rises through the sensor module 20, and the concentration of the corresponding gaseous phase materials may be calculated through the potential value.

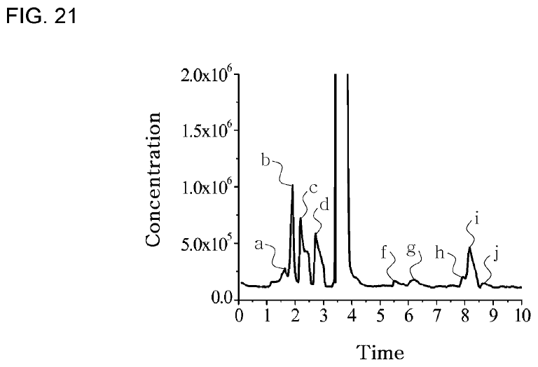

FIG. 21 is a graph showing the detection results through the measuring system 10 when the plasma cleaning process is performed.

As shown in FIG. 21, it can be seen that a sharp peak or a remarkable rise in potential value appears at the predetermined time.

The substances of the gaseous phase materials detected at the corresponding time are already identified. For example, the material of peak a is pyridine, and the material of peak b is butanediol.

Additionally, the substances and concentration of the gaseous phase materials 60 in the gas mixture 6 from the treatment apparatus 1 may be analyzed through the potential value by the substances of the corresponding gaseous phase materials.

The worker or the control unit of the system may continuously identify the presence or absence of residue on the cleaned wafer 3 by identifying the substances and concentration of the detected gaseous phase materials 60. When the concentration of the specific material is close to 0 or below a predetermined reference value, the worker or the control unit of the system may determine that there is no residue on the cleaned wafer 3 and terminate the cleaning operation.

According to the above-described configuration, it is possible to identify the presence or absence of residue by identifying the presence and concentration of the gaseous phase materials 60 included in the gas mixture 6 from the treatment apparatus 1 while the plasma cleaning process is being performed (in-situ). Accordingly, it is possible to omit the process including stopping the process, testing the wafer using an extra device, and when the test results are below the reference, performing the cleaning process again, thereby significantly improving the yield of the semiconductor component.

Additionally, since the sum of the sampling time and the desorption time and the separation time of the gaseous phase materials 60 from the concentration module is much shorter than the time taken to stop the treatment apparatus 1, transfer the wafer, test and treat again, it is possible to identify the processed state of the wafer substantially “in real time”.

### Other Embodiment

Although the treatment apparatus 1 is described as a wafer plasma cleaning apparatus in the above-described description, the treatment apparatus 1 is not limited thereto. The treatment apparatus 1 may be a UV ozone based cleaning apparatus. Additionally, the treatment apparatus 1 may be a plasma, UV ozone based wafer etching apparatus. The etching method may be a dry method or a dry method. Additionally, the component being treated is not limited to the wafer and may include any other semiconductor component, and may not be a semiconductor component. The measuring system 10 according to this embodiment may be applied to any process in which the gas mixture is produced by reaction between reactive gases and the component in the chamber.

Furthermore, the treatment apparatus 1 is not limited to an apparatus that performs a specific “process”. For example, the measuring system 10 may be installed at a chimney as “the pipe 20” through which exhaust gases from a factory are released, in order to meter the components of the exhaust gases. Additionally, the treatment apparatus 1 may be any other apparatus that emits gas mixtures, for example, vehicles.

Additionally, according to the above-described embodiment, the detection unit 12 includes the single portable detection device 120, but is not limited thereto. The concentration module 200, the separation module 300 and the sensor module 400 of the detection unit 12 may be respective devices, and each device may not be small enough to carry.

Additionally, according to the above-described embodiment, a PID type sensor is used in the sensor module 400, but a sensor using flame ionization detection (FID) may be used.

Additionally, in the above-described embodiment, the sampler 103 using the spiral conduit is used, but is not limited thereto. The sampler may include, for example, simply a linear conduit shape or a sample bag, to temporarily store the gas mixture 6 as much as the predetermined volume.

Additionally, according to the above-described embodiment, the sampling unit 11 and the detection unit 12 are in fluid communication with each other, but is not limited thereto. After sampling is performed by the sampling unit 11, the sampling unit 11 may be separated, transferred and connected to the detection unit 12 of a different location.

According to the above-described embodiment, the valve assembly 500, 500′ is connected to the pipe 20 in a bypassed manner through the upstream connection pipe 31 and the downstream connection pipe 32 branched from the pipe 20, but is not limited thereto. By the deletion of the conduit between the upstream connection pipe 31 and the downstream connection pipe 32 in FIG. 1, the gas mixture 6 passing through the pipe 20 may pass through the valve assembly 500, 500′.

What is claimed is:

1. A measuring system for metering components of a gas mixture including gaseous phase materials, the measuring system comprising:
a sampling unit configured to selectively communicate with a pipe through which the gas mixture passes to sample the gas mixture from the pipe;
a detection unit configured to separate and detect the gaseous phase materials included in the gas mixture sampled by the sampling unit into the components;
a valve assembly including a plurality of valves to selectively bring the sampling unit into communication with the pipe or the detection unit,
wherein the plurality of valves cooperates to selectively perform a sampling connection operation of bringing the pipe and the sampling unit into communication with each other and a sampling interruption operation of interrupting the communication between the pipe and the sampling unit.

2. The measuring system according to claim 1, wherein the plurality of valves cooperates to perform a gas delivery operation of forming a flow channel to allow a carrier gas to flow with the gas mixture, simultaneously with the sampling interruption operation or after the sampling interruption operation.

3. The measuring system according to claim 2, wherein the flow channel is formed by the gas delivery operation to allow the carrier gas to flow to the detection unit through the sampling unit.

4. The measuring system according to claim 2, wherein in the sampling connection operation, the channel of fluid flow from a carrier gas tank directly to the detection unit and the channel of fluid flow from the pipe back to the pipe through the sampling unit are formed, and
wherein in the gas delivery operation, the channel of fluid flow from the carrier gas tank to the detection unit through the sampling unit is formed.

5. The measuring system according to claim 2, wherein the sampling interruption operation includes:
a sampling route closing operation of trapping the gas mixture in the sampling unit, and
a depressurization operation of reducing a pressure of the gas mixture trapped in the sampling unit.

6. The measuring system according to claim 5, wherein the detection unit includes a depressurization chamber to reduce the pressure of the gas mixture,
wherein the gas mixture is allowed to enter the depressurization chamber to reduce the pressure through the depressurization operation, and
wherein after the pressure of the gas mixture is reduced, the gas delivery operation is performed.

7. The measuring system according to claim 2, wherein the plurality of valves includes a plurality of multi-port valves, each having a plurality of ports, and
wherein the plurality of multi-port valves selectively operates to selectively perform the sampling connection operation, the sampling interruption operation and the gas delivery operation.

8. The measuring system according to claim 7, wherein each of the plurality of multi-port valves is a manual valve that switches a fluid channel in the valve by a lever that is manually manipulated, and
wherein each lever of the plurality of multi-port valves is connected to a link structure together, and the levers of the plurality of multi-port valves are simultaneously operated by operation of the link structure to simultaneously switch the fluid channels.

9. The measuring system according to claim 7, wherein the plurality of multi-port valves includes first to third 3-way valves, each having three ports,
wherein a first port of the first 3-way valve is connected to the pipe, a second port is connected to an end of a first connection pipe, and a third port is connected to the pipe,
wherein a first port of the second 3-way valve is connected to an entrance of the sampling unit, a second port is connected to an opposite end of the first connection pipe, and a third port is connected to an end of a second connection pipe,
wherein a first port of the third 3-way valve is connected to an exit of the sampling unit, a second port is connected to the pipe, and a third port is connected to an end of a third connection pipe, and
wherein the sampling unit and the pipe are brought into communication with each other or the communication is interrupted by operation of the first 3-way valve.

10. The measuring system according to claim 9, wherein the plurality of multi-port valves includes a fourth 3-way valve and a fifth 3-way valve, each having three ports,
wherein a first port of the fourth 3-way valve is connected to a carrier gas inlet pipe that communicates with a carrier gas tank, a second port is connected to an opposite end of the second connection pipe, and a third port is connected to an end of a fourth connection pipe,
wherein a first port of the fifth 3-way valve is connected to a detection unit inlet pipe that communicates with the detection unit, a second port is connected to an opposite end of the fourth connection pipe, and a third port is connected to an opposite end of the third connection pipe, and
wherein the first to fifth 3-way valves selectively operate to switch the fluid channel to selectively perform the sampling connection operation, the sampling interruption operation and the gas delivery operation.

11. The measuring system according to claim 10, wherein after a pressure of the gas mixture is reduced through the sampling interruption operation, the gas delivery operation is performed,
wherein the sampling interruption operation includes a sampling route closing operation of trapping the gas mixture in the sampling unit and a depressurization operation of allowing the gas mixture trapped in the sampling unit to flow to a depressurization chamber to reduce the pressure of the gas mixture,
wherein in the sampling route closing operation, the first 3-way valve and the third 3-way valve simultaneously operate to switch the fluid channel, and
wherein in the depressurization operation, the fifth 3-way valve operates to switch the fluid channel.

12. The measuring system according to claim 11, wherein in the gas delivery operation, the second 3-way valve and the fourth 3-way valve simultaneously operate to switch the fluid channel.

13. The measuring system according to claim 10, wherein the gas delivery operation is performed simultaneously with the sampling interruption operation, and
wherein the first to fifth 3-way valves simultaneously operate to switch the fluid channel to simultaneously perform the sampling interruption operation and the gas delivery operation.

14. The measuring system according to claim 7, wherein the plurality of multi-port valves includes a first 4-way valve and a second 4-way valve, each having four ports,
wherein a first port of the first 4-way valve is connected to a carrier gas inlet pipe that communicates with a carrier gas tank, a second port is connected to an entrance of the sampling unit, a third port is connected to the pipe, and a fourth port is connected to an end of a fifth connection pipe,
wherein a first port of the second 4-way valve is connected to a detection unit inlet pipe that communicates with the detection unit, a second port is connected to an exit of the sampling unit, a third port is connected to the pipe, and a fourth port is connected to an opposite end of the fifth connection pipe,
wherein the first 4-way valve and the second 4-way valve simultaneously operate to switch the fluid channel,
wherein in the sampling connection operation, the channel of fluid flow from the carrier gas tank directly to the detection unit and the channel of fluid flow from the pipe back to the pipe through the sampling unit are formed, and
wherein in the gas delivery operation, the channel of fluid flow from the carrier gas tank to the detection unit through the sampling unit is formed.

15. The measuring system according to claim 14, wherein the first 4-way valve and the second 4-way valve are an automatic valve that automatically operates by control of a controller.
